(19) United States
(12) Patent Application Publication
(10) Pub. No.: US 2024/0225760 A9
(43) Pub. Date: Jul. 11, 2024

(54) SYSTEMS AND METHODS FOR DETERMINING A SAFETY LAYER FOR AN ANATOMICAL ELEMENT

(71) Applicant: Mazor Robotics Ltd., Caesarea (IL)

(72) Inventors: Itamar Eshel, Tzur Igal (IL); Hay Shmulevich, Shaked (IL); Jaffar Hleihil, Zichron Yaakov (IL); Dvir Kadshai, Tel Aviv (IL)

(21) Appl. No.: 17/972,191

(22) Filed: Oct. 24, 2022

(51) Int. Cl. A61B 34/32

(52) U.S. Cl. CPC A61B 34/32; A61B 2034/305

(57) ABSTRACT

Systems and methods for determining a safety layer for an anatomical element are provided. A cutting tool contacting the anatomical element at a first surface may be detected and dimensional information about the anatomical element may be received. A safety layer may be determined for the anatomical element based on the dimensional information and the detected contact between the cutting tool and the anatomical element at the first surface.

### BACKGROUND

The present disclosure is generally directed to determining safety layers or boundaries, and relates more particularly to determining a safety layer for an anatomical element during a cutting process.

Surgical robots may assist a surgeon or other medical provider in carrying out a surgical procedure, or may complete one or more surgical procedures autonomously. During such surgical procedures, surgical tools may be used on one or more anatomical elements. The tools may be oriented and operated by the surgical robot and/or the surgeon or other medical provider.

### BRIEF SUMMARY

Example aspects of the present disclosure include:

A system for determining a safety layer for an anatomical element according to at least one embodiment of the present disclosure comprises a processor; and a memory storing data for processing by the processor, the data, when processed, causes the processor to: detect a cutting tool contacting the anatomical element at a first surface; receive dimensional information of the anatomical element; and determine a safety layer for the anatomical element based on the dimensional information and the detected contact between the cutting tool and the anatomical element at the first surface, wherein the cutting tool is prevented from moving past the safety layer.

Any of the aspects herein, wherein the memory stores further data for processing by the processor that, when processed, causes the processor to receive information describing a first pose corresponding to a moment when the cutting tool contacts the anatomical element.

Any of the aspects herein, wherein determining the safety layer comprises determining the distance from the first pose to a second pose based on the dimensional information, the second pose corresponding to when the cutting tool breaches the anatomical element and spacing the safety layer a predetermined distance from the second pose towards the first pose.

Any of the aspects herein, wherein the first pose is received from a robot operating the cutting tool.

Any of the aspects herein, wherein determining the safety layer comprises determining a second surface opposite the first surface based on the dimensional information and spacing the safety layer a predetermined distance from the second surface towards the first surface.

Any of the aspects herein, wherein the predetermined distance is substantially constant throughout the safety layer.

Any of the aspects herein, wherein the predetermined distance is variable through at least a portion of the safety layer.

Any of the aspects herein, wherein spacing the safety layer a predetermined distance from the second surface includes multiplying a distance between the second surface and the first surface by a predetermined safety factor.

Any of the aspects herein, wherein a sensor output is used as part of detecting the cutting tool contacting the anatomical element.

Any of the aspects herein, wherein the sensor output comprises a force measured by a force sensor in response to the cutting tool exerting the force on the force sensor, and wherein the cutting tool contacting the anatomical element is detected when the measured force exceeds a threshold force.

Any of the aspects herein, wherein the dimensional information comprises a three-dimensional model of the anatomical element.

Any of the aspects herein, wherein the memory stores further data for processing by the processor that, when processed, causes the processor to generate a notification when the cutting tool contacts the safety layer.

A system for determining a safety layer for an anatomical element according to at least one embodiment of the present disclosure a sensor configured to measure a parameter to yield a sensor output; a processor; and a memory storing data for processing by the processor, the data, when processed, causes the processor to: detect a cutting tool contacting the anatomical element at a first surface using a sensor output received from the sensor; receive dimensional information of the anatomical element; determine a safety layer for the anatomical element based on the dimensional information and the detected contact between the cutting tool and the anatomical element at the first surface, wherein the cutting tool is prevented from moving past the safety layer; and generate a notification when the cutting tool contacts the safety layer.

Any of the aspects herein, wherein the memory stores further data for processing by the processor that, when processed, causes the processor to: receive information describing a first pose corresponding to a moment when the cutting tool contacts the anatomical element.

Any of the aspects herein, wherein determining the safety layer comprises determining the distance from the first pose to a second pose based on the dimensional data, the second pose corresponding to when the cutting tool breaches the anatomical element and spacing the safety layer a predetermined distance from the second pose towards the first pose.

Any of the aspects herein, further comprising a robotic arm configured to orient the cutting tool, wherein the first pose is received from the robot.

Any of the aspects herein, wherein determining the safety layer comprises determining a second surface opposite the first surface based on the dimensional data and spacing the safety layer a predetermined distance from the second surface towards the first surface.

Any of the aspects herein, wherein the sensor output comprises a force measured by a force sensor in response to the cutting tool exerting the force on the force sensor, and wherein the cutting tool contacting the anatomical element is detected when the measured force exceeds a threshold force.

Any of the aspects herein, wherein the dimensional information comprises a three-dimensional model of the anatomical element.

A system for determining a safety layer for an anatomical element according to at least one embodiment of the present disclosure a robotic arm configured to hold and orient a cutting tool relative to the anatomical element; a processor; and a memory storing data for processing by the processor, the data, when processed, causes the processor to: cause the robotic arm to orient the cutting tool on a trajectory; detect when the cutting tool contacts the anatomical element at a first surface; receive dimensional information of the anatomical element; determine a safety layer for the anatomical element based on the dimensional information and the detected contact, wherein the cutting tool is prevented from moving past the safety layer; and cause the robotic arm to stop orienting the cutting tool on the trajectory when the cutting tool reaches the safety layer.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2A is an illustration of a cutting plane on an anatomical element according to at least one embodiment of the present disclosure;

FIG. 2B is a detailed illustration of a cutting plane on an anatomical element according to at least one embodiment of the present disclosure;

FIG. 3 is a schematic illustration of a cutting tool and an anatomical element according to at least one embodiment of the present disclosure;

FIG. 4 is a force vs. depth graph according to at least one embodiment of the present disclosure; and

FIG. 5 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Robotic assisted surgery or autonomous robotic surgery enable autonomous surgical procedures such as, for example, autonomous bone removal. When performing robotic or robotic assisted bone removal, one risk includes breaching the anatomical element upon which the cutting and removal is being performed upon. For example, in embodiments where the anatomical element comprises an anterior cortical bone, breach of such bone by a cutting instrument exposes a patient's spinal cord and/or nerves to damage by the cutting instrument. Thus, it is desirable to have safety measures to mitigate such risk. Some methods for avoiding a breach include detecting an actual breach of the bone. However, detecting actual breach introduces risks to the patient as sensitive anatomical matter is exposed to the cutting instrument during an actual breach.

In at least one embodiment of the present disclosure, an additional layer of safety is provided using a predictive safety layer based on a combination of a detection of a first contact between a cutting tool and an anatomical element (e.g., bone) and dimensional information such as, for example, three-dimensional (3D) data of the anatomical element. A system could use such information to stop the cutting tool before the cutting tool reaches a no-fly zone. More specifically, by combining the information of a time and/or pose of a first contact between the cutting tool and the anatomical element with a maximum depth allowed (taken from, for example, the 3D data), an algorithm can be developed to allow the system another condition for stopping the cutting tool as a safety measure. This algorithm can, for example, tell the system to stop cutting after a distance of L*0.8 (or any other factor of safety) or after a breach of the anatomical element has been detected (or whichever comes first). The distance L may define a cutting length at the point of penetration or first contact by the cutting tool to the anatomical element to a point of breach by the cutting tool.

Embodiments of the present disclosure provide technical solutions to one or more of the problems of (1) preventing breach of an anatomical element by a cutting tool during a cutting procedure, (2) protecting sensitive anatomical elements during a cutting procedure, and (3) increasing patient safety.

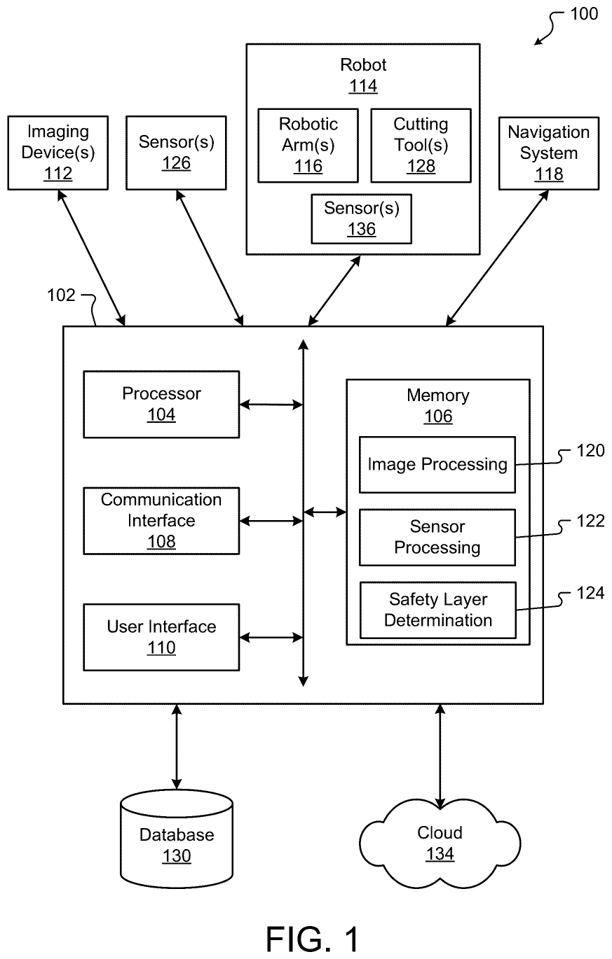

Turning first to FIG. 1, a block diagram of a system 100 according to at least one embodiment of the present disclosure is shown. The system 100 may be used to generate one or more safety measures and/or carry out one or more other aspects of one or more of the methods disclosed herein. The system 100 comprises a computing device 102, one or more imaging devices 112, a robot 114, a navigation system 118, one or more sensors 126, a database 130, and/or a cloud or other network 134. Systems according to other embodiments of the present disclosure may comprise more or fewer components than the system 100. For example, the system 100 may not include the imaging device 112, the robot 114, the navigation system 118, one or more sensors 126, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the sensors 126, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the method 500 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, sensor processing 122, and/or safety layer determination 124. Such content, if provided as in instruction, may, in some embodiments, be organized into one or more applications, modules, packages, layers, or engines.

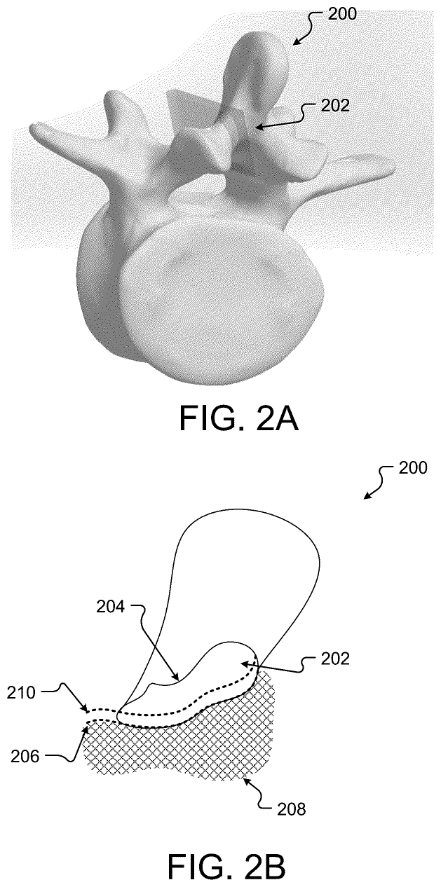

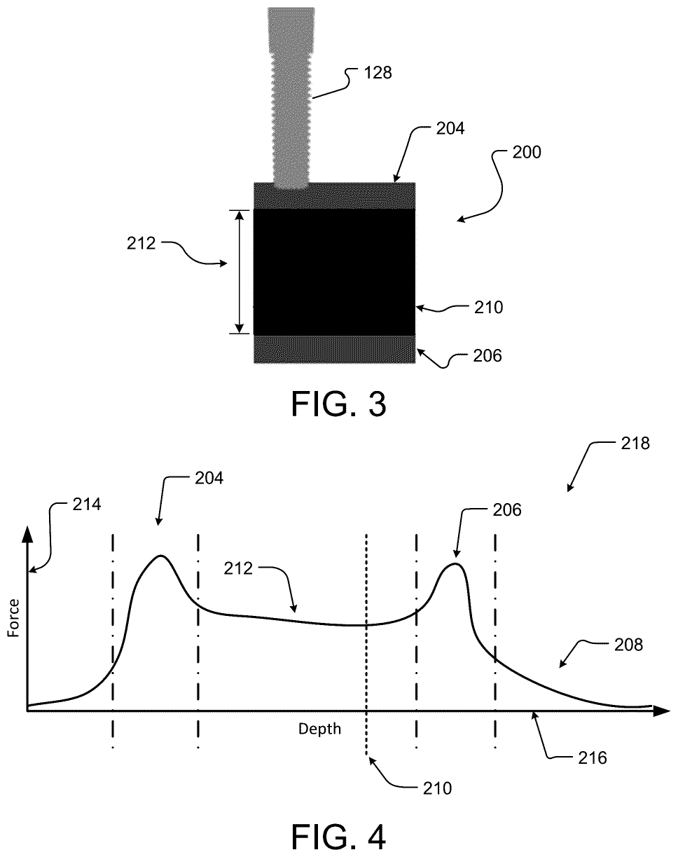

The image processing 120 enables the processor 104 to process image data of an image (received from, for example, the imaging device 112, an imaging device of the navigation system 118, or any imaging device) for the purpose of, for example, identifying information about an anatomical element 200 (shown in FIGS. 2 and 3) and/or objects in the image such as a cutting tool 128. The identifying information can be used to determine when the cutting tool has first contacted an anatomical element and/or a pose of the cutting tool at such contact. It will be appreciated that alternatively or additionally the first contact can be determined from sensor output from the one or more sensors 126, as described below.

The sensor processing 122 enables the processor 104 to process sensor output data (received from for example, the one or more sensors 126) for the purpose of, for example, determining first contact between the cutting tool 128 and the anatomical element 200. The sensor output may be received as signal(s) and may be processed by the processor 104 using the sensor processing 122 to output data such as, for example, force data, acceleration data, pose data, time data, etc.

The safety layer determination 124 enables the processor 104 to process inputs such as information about a first contact between the cutting tool 128 and the anatomical element 200 and information about the anatomical element 200 for the purpose of, for example, generating a safety layer. Information about the first contact may include a pose of the cutting tool 128 and/or a time stamp corresponding to when the cutting tool 128 first contacts the anatomical element 200. The information about the anatomical element 200 may include a 3D model and/or 2D images of the anatomical element 200, dimensional information about the anatomical element 200, a volume of the anatomical element 200, and/or a pose of the anatomical element 200 relative to the patient and/or the cutting tool 128. The processor 104 may output a safety layer or boundary that may restrict or prevent the cutting tool 128 from advancing past the safety layer or boundary. The safety layer or boundary may beneficially prevent breach of an anatomical element by the cutting tool 128 by preventing the cutting tool 128 from drilling or cutting past the safety layer.

Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, they sensors 126, and/or the cloud 134.

The computing device 102 may also comprise a communication interface 108. The communication interface 108 may be used for receiving image data or other information from an external source (such as the imaging device 112, the robot 114, the navigation system 118, the sensors 126, the database 130, the cloud 134, and/or any other system or component not part of the system 100), and/or for transmitting instructions, images, or other information to an external system or device (e.g., another computing device 102, the imaging device 112, the robot 114, the navigation system 118, the sensors 126, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy and/or objects such as the cutting tool 128 to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof, and/or objects such as the cutting tool 128. The image data may be or comprise a preoperative image, an intraoperative image, a postoperative image, or an image taken independently of any surgical procedure. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (Mill) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient and/or objects such as the cutting tool 128.

The sensors 126 can be configured to provide sensor output. The sensors 126 can comprise a force sensor configured to detect a force applied on the robotic arm 116 (e.g., whether via an end effector of the robotic arm 116, the cutting tool 128 held by an end effector of the robotic arm 116, or otherwise) or applied by the cutting tool 128 (e.g., whether held or supported by the robotic 116 or by a user such as a surgeon or other medical provider). In other embodiments, the sensors 126 may alternatively or additionally comprise a position sensor, a proximity sensor, a magnetometer, or an accelerometer. In some embodiments, the sensor 126 may be a linear encoder, a rotary encoder, or an incremental encoder. In still other embodiments, the sensor 126 may be an imaging sensor. Other types of sensors may also be used as the sensor 126.

Sensor output or output data from the sensors 126 may be provided to a processor of the robot 114, to the processor 104 of the computing device 102, and/or to the navigation system 118. Output data from the sensor(s) 126 may also be used to determine when the cutting tool 128 initially contacts the anatomical element 200. For example, the output data may include force data, which can be used by the processor 104 (or a processor of the robot 114) to determine when the cutting tool 128 contacts the anatomical element 200, as will described in more detail in FIG. 4.

It will be appreciated that in some embodiments, the sensors 126 may be a component separate from the robotic arm(s) 116. In other embodiments, sensor 136—which may be the same as or similar to the sensors 126—may be integrated with the robot 114. In such embodiments, the sensor 136 may enable the processor 104 (or a processor of the robot 114) to determine a precise pose in space of a robotic arm 116 (as well as any object or element held by or secured to the robotic arm). In other words, sensor output or output data from the sensor 136 may be used to calculate a position in space of the robotic arm 116 (and thus, the cutting tool 128) relative to one or more coordinate systems.

The robot 114 may be any surgical robot or surgical robotic system. The robot 114 may be or comprise, for example, the Mazor X™ Stealth Edition robotic guidance system. The robot 114 may be configured to position the cutting tool 128 at one or more precise poses (e.g., position(s) and orientation(s)). The cutting tool 128 may be any tool capable of cutting, drilling, milling, and/or parting an anatomical element. The cutting tool 128 may be, for example, a drill bit. In some embodiments, the robot 114 may be configured to rotate and/or advance the cutting tool 128 using, for example, one or more motors to rotate the cutting tool 128.

The robot 114 may additionally or alternatively be configured to manipulate any component (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the cutting tool 128. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, a cutting tool 128, surgical tool, or other object held by the robot 114 (or, more specifically, by the robotic arm 116) may be precisely positionable in one or more needed and specific positions and orientations.

In some embodiments, reference markers (e.g., navigation markers) may be placed on the robot 114 (including, e.g., on the robotic arm 116), the cutting tool 128, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., the cutting tool 128) and the system can operate without the use of the robot 114 (e.g., with the surgeon manually manipulating the cutting tool 128 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, the cutting tool 128), and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of the method 500 described herein. The system 100 or similar systems may also be used for other purposes.

FIG. 2A illustrates the anatomical element 200 and a cutting plane 202. FIG. 2B illustrates a cross-sectional view of the anatomical element at the cutting plane 202. In the illustrated embodiment, a cross-sectional view of the anatomical element 200 is shown where the anatomical element 200 comprises a bone, and more specifically, a vertebra. In other embodiments, the anatomical element 200 may be any bone, organ, or anatomical feature of a patient. As previously described, during a cutting process of the anatomical element 200 using the cutting tool 128, it may be desirable to stop the cutting tool 128 prior to breach of the anatomical element 200. The cutting plane 202 may define a first surface 204 and a second surface 206 opposite the first surface 204. In some embodiments, the second surface 206 may represent the start of a no-fly zone 208. In other words, the second surface 206 may be the surface at which the cutting tool 128 may breach the anatomical element 200 and may enter or contact sensitive anatomical matter. For example, the anatomical element 200 may comprise a vertebra, the first surface 204 may correspond to a posterior cortical through which the cutting tool 128 first contacts the vertebrae, the second surface 206 may correspond to an anterior cortical, and the no-fly zone 208 may correspond to the spinal cord and/or nerves. Thus, it would be undesirable for the cutting tool 128 to breach the anterior cortical and enter the no-fly zone 208 in which the cutting tool 128 may damage the spinal cord and/or nerves. As such, embodiments of the present disclosure provide for determining a safety layer 210 to prevent the cutting tool 128 from breaching or contacting the second surface 206 and thus, also prevent the cutting tool 128 from entering the no-fly zone 208.

It will be appreciated that the cutting plane 202, the second surface 206 (as represented by a dotted line), the no-fly zone 208 (as represented by cross-hatching), and the safety layer 210 are shown for illustrative purposes in FIG. 2B. However, in some instances, the cutting plane 202, the second surface 206, the no-fly zone 208, and/or the safety layer 210 may be illustrated on a display or a user interface such as the user interface 110.

FIG. 3 is a schematic illustration of the cutting tool 128 contacting the anatomical element 200. As shown and previously described the cutting tool 128 contacts the anatomical element 200 at the first surface 204. The cutting tool 128 may advance through the anatomical element 200 until the cutting tool 128 reaches the safety layer 210. The safety layer 210 may be determined based on information about the cutting tool 128 when the cutting tool 128 contacts the anatomical element 200 at the first surface 204 and dimensional information about the anatomical element 200 such as, for example, a distance 212 (e.g., a distance “L” as previously described) between the first surface 204 and the second surface 206, a pose of the first surface 204 and/or the second surface 206, a volume of the anatomical element 200, a shape of the anatomical element 200, or the like. The distance 212 may define a cutting length at the point of penetration or first contact by the cutting tool to the anatomical element to a point of breach by the cutting tool. The dimensional information may comprise a 3D model of the anatomical element 200, 2D image(s) of the anatomical element 200, or information (whether in machine-readable form or human readable form) about the anatomical element 200 such as dimensions (e.g., width, height, volume, etc.). As the cutting tool 128 advances towards the second surface 206, the safety layer 210 may cause the cutting tool 128 to stop (whether by causing a robotic arm such as the robotic arm 116 to stop movement or by preventing a user such as a surgeon from advancing the cutting tool 128) and/or may generate a notification that the cutting tool 128 has reached the safety layer 210. In some instances, the cutting tool 128 may advance past the safety layer 210 and the cutting tool 128 may be stopped or a notification may be generated when the cutting tool 128 breaches the second surface 206.

FIG. 4 illustrates a force 214 vs. a depth 216 graph 218 which represents the force exerted by the cutting tool 128 on, for example, the sensor 126, and a depth of cut of the cutting tool 128. As illustrated, by measuring the force exerted by the cutting tool 128, the first contact by the cutting tool 128 at the first surface 204 can be detected. It will be appreciated that in other embodiments, the first contact by the cutting tool 128 can be measuring in other ways such as, for example, visual detection, using an electronic gyroscope, and/or measuring electric conductivity. Electric conductivity can be used to measure the first contact by sensing a change in conductivity between different layers of the anatomical element 200. The change in conductivity can be measured by using a transceiver and a receiver to send electronic pulses through the anatomical element 200 and measuring the signal. As shown, the force may increase when the cutting tool 128 contacts and/or is cutting through the first surface 204 and when the cutting tool 128 contacts and/or is cutting through the second surface 206. This is due to the higher bone density in the cortical regions of the anatomical element 200 (e.g., the vertebra) and the lower bone density in the cancellous region of the anatomical element 200. Thus, the first contact between the cutting tool 128 and the first surface 204 may be detected by monitoring the force exerted by the cutting tool 128 (by, for example, the sensor(s) 126). In some instances, when the force exerted meets or exceeds a threshold force, information about the first contact may be measured, recorded, and/or a notification may be generated indicating the first contact. For example, a first pose of the cutting tool 128 may be measured and/or recorded when the first contact is detected. As further shown in the illustrated graph (and also in FIGS. 2 and 3), the safety layer 210 may be spaced a predetermined distance from the second surface 206 towards the first surface 204. The determination of this spacing is described in further detail below.

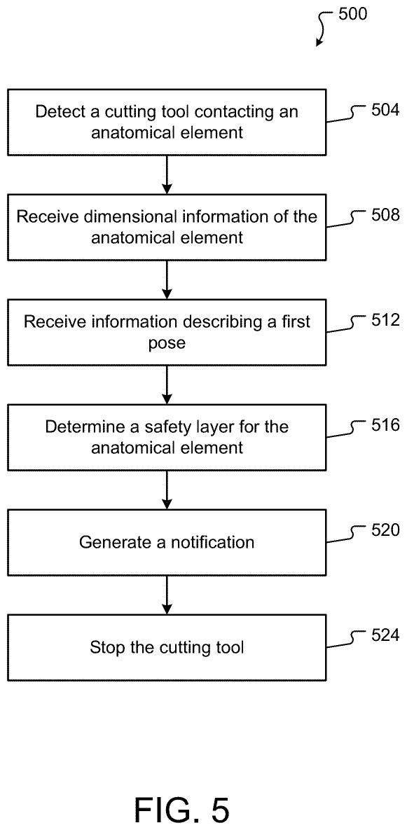

FIG. 5 depicts a method 500 that may be used, for example, for determining a safety layer for an anatomical element.

The method 500 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 500. The at least one processor may perform the method 500 by executing elements stored in a memory such as the memory 106. The elements stored in memory and executed by the processor may cause the processor to execute one or more steps of a function as shown in method 500. One or more portions of a method 300 may be performed by the processor executing any of the contents of memory, such as an image processing 120, a sensor processing 122, and/or a safety layer determination 124.

The method 500 comprises detecting a cutting tool contacting an anatomical element (step 504). The cutting tool may be the same as or similar to the cutting tool 128 and the anatomical element may be the same as or similar to the anatomical element 200. Detecting when the cutting tool contacts the anatomical element may detect when the cutting tool contacts the anatomical element at a first surface such as the first surface 204. In some embodiments, the contact may be detected using a sensor such as the sensor 126 configured to measure and yield sensor output and a processor such as the processor 104 configured to execute a sensor processing such as the sensor processing 122 to process the sensor output. The sensor processing enables the processor to process the sensor output data for the purpose of, for example, determining first contact between the cutting tool and the anatomical element. The sensor output may be received as signal(s) and may be processed by the processor using the sensor processing to output data such as, for example, force data, acceleration data, pose data, time data, etc.

The sensor may comprise, for example, a force sensor configured to measure a force exerted by the cutting tool. In such embodiments, the force sensor may be used to monitor the force and the detected contact (e.g., the first contact between the cutting tool and the anatomical element) may correspond to when the measured force exceeds a threshold force. The threshold force may be determined automatically using artificial intelligence and training data (e.g., historical cases) in some embodiments. In other embodiments, the threshold force may be or comprise, or be based on, surgeon input received via the user interface. In further embodiments, the threshold force may be determined automatically using artificial intelligence, and may thereafter be reviewed and approved (or modified) by a surgeon or other user. In other embodiments, the contact may be detected optically by, for example, using an imaging device such as the imaging device 112 or by measuring an electric conductivity. In still other embodiments, the contact may be predicted based on a surgical plan and the contact may be confirmed using the sensors, imaging, and/or electric conductivity.

In some embodiments, the cutting tool is oriented and operated by a robotic arm such as the robotic arm 116 of a robot such as the robot 114. In such embodiments, the robotic arm can automatically orient the cutting tool along a trajectory. In other embodiments, the robotic arm can assist a user (such as, for example, a surgeon) in orienting and/or operating the cutting tool. In still other embodiments, the cutting tool may be oriented and/or operated by the user.

The method 500 also comprises receiving dimensional information of the anatomical element (step 508). The dimensional information may be received from, for example, a communication interface such as the communication interface 108, the imaging device, a user interface such as the user interface 110, a memory such as the memory 106, a database such as the database 130, and/or a cloud such as the cloud 134. The dimensional information may comprise, for example, a distance such as the distance 212 between the first surface and a second surface such as the second surface 206 opposite the first surface, a pose of the first surface and/or the second surface, a volume of the anatomical element, a shape of the anatomical element, or the like. The dimensional information may also comprise a 3D model of the anatomical element, 2D image(s) of the anatomical element, or information (whether in machine-readable form or human readable form) about the anatomical element such as dimensions (e.g., width, height, volume, etc.). In embodiments where a 3D model(s) or 2D image(s) are received, the processor may execute an image processing such as the image processing 120 to process the 3D model(s) or 2D image(s) to measure and/or extract dimensional information about the anatomical element.

The method 500 also comprises receiving information describing a first pose of the cutting tool (step 512). The first pose may correspond to a moment when the cutting tool contacts the anatomical element. The information describing the first pose may be received from the robotic arm in instances where the robotic arm is configured to orient and/or operate the cutting tool or is configured to support the user in orienting and/or operating the cutting tool. In other instances, the information describing the first pose may be determined from 3D model(s) or 2D image(s) taken by, for example, the imaging device. In still other instances, the information describing the first pose may be received from a navigation system such as the navigation system 118.

It will be appreciated that in some embodiments, the method 500 may not include the step 512.

The method 500 also comprises determining a safety layer for the anatomical element (step 516). Determining the safety layer may include using the processor to execute a safety layer determination such as the safety layer determination 124 to generate the safety layer. The safety layer determination enables the processor to process inputs such as information about a first contact between the cutting tool and the anatomical element, input from a user such as a surgeon or other medical provider, and information about the anatomical element for the purpose of, for example, generating the safety layer. Information about the first contact may include a first pose of the cutting tool and/or a time stamp corresponding to when the cutting tool first contacts the anatomical element. The input from the user may comprise a safety factor used to determine the safety layer. The information about the anatomical element (as described in the step 508 above) may include a 3D model and/or 2D images of the anatomical element, dimensional information about the anatomical element, a volume of the anatomical element, and/or a pose of the anatomical element relative to the patient and/or the cutting tool. The processor may output a safety layer or boundary that may restrict or prevent the cutting tool from advancing past the safety layer or boundary.

More specifically, in some embodiments, determining the safety layer may comprise determining a distance between the first pose (e.g., a pose of the cutting tool corresponding to the moment when the cutting tool contacts the anatomical element) and a second pose (e.g., a pose of the cutting tool corresponding to when the cutting tool breaches the anatomical element) based on the dimensional information. The safety layer in such embodiments may be spaced a predetermined distance from the second pose towards the first pose. Such spacing may be determined by multiplying the distance (which can be the same as or similar to the distance 212) by a predetermined safety factor, which can be received as input from the user. In some embodiments the predetermined safety factor may be, for example 0.8, though in other embodiments the predetermined safety factor may be greater than or less than 0.8.

In other embodiments, determining the safety layer may comprise determining the second surface based on the dimensional information and spacing the safety layer a predetermined distance from the second surface towards the first surface. In such embodiments, the predetermined distance may be substantially constant throughout the safety layer. Alternatively, the predetermined distance may be variable through at least a portion of the safety layer. In at least some embodiments, spacing the safety layer the predetermined distance from the second layer may include multiplying or adding a distance between the second surface and the first surface by a predetermined safety factor (whether by a constant or a percentage). It will be appreciated that the predetermined safety factor may be received as input from the user. In some embodiments the predetermined safety factor may be, for example 0.8, though in other embodiments the predetermined safety factor may be greater than or less than 0.8.

The method 500 also comprises generating a notification (step 520). The notification may be generated when one or more conditions are met such as, for example, when contact between the cutting tool and the anatomical element is detected, when the cutting tool meets or exceeds the safety layer, and/or when the cutting tool breaches the anatomical element. The notification may alert the user of one or more conditions and may also cause the cutting tool to cease or stop cutting as described in step 524 below. The notification may be a visual notification, an audible notification, or any type of notification communicated to a user. The notification may be communicated to the user via the user interface. In some embodiments, the notification may be automatically generated by the processor. In other embodiments, the notification may be automatically generated by any component of a system such as the system 100.

It will be appreciated that in some embodiments, the method 500 may not include the step 520.

The method 500 also comprises stopping the cutting tool (step 524). In embodiments where the cutting tool is oriented and/or operated automatically by the robotic arm, stopping the cutting tool may include causing the robotic arm to stop orienting the cutting tool on the trajectory and/or stop operation of the cutting tool. In embodiments where the robotic arm may assist the user in orienting and/or operating the cutting tool and/or where the user is manually orienting and/or operating the cutting tool, stopping the cutting tool may include preventing the user from advancing the cutting tool (whether by causing the robotic arm to stop movement, alerting and/or instructing the user to stop movement, etc.).

Stopping the cutting tool (whether automatically or manually) may be triggered or occur when the cutting tool has reached or passed the safety layer. The safety layer enables automatic cutting of the anatomical element by the cutting tool to a position that is spaced a safe distance away from breaching the anatomical element. Thus, the safety layer may reduce the time needed to perform a portion of a cutting procedure and may enable positioning the cutting tool in a safe position relative to a no-fly zone. In such instances, the user may be able to manually orient and operate the cutting tool past the safety layer to complete a cutting procedure.

The present disclosure encompasses embodiments of the method 500 that comprise more or fewer steps than those described above, and/or one or more steps that are different than the steps described above.

As noted above, the present disclosure encompasses methods with fewer than all of the steps identified in FIG. 5 (and the corresponding description of the method 500), as well as methods that include additional steps beyond those identified in FIG. 5 (and the corresponding description of the method 500). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for determining a safety layer for an anatomical element comprising:
a processor; and
a memory storing data for processing by the processor, the data, when processed, causes the processor to:
detect a cutting tool contacting the anatomical element at a first surface;
receive dimensional information of the anatomical element; and
determine a safety layer for the anatomical element based on the dimensional information and the detected contact between the cutting tool and the anatomical element at the first surface, wherein the cutting tool is prevented from moving past the safety layer.

2. The system of claim 1, wherein the memory stores further data for processing by the processor that, when processed, causes the processor to:
receive information describing a first pose corresponding to a moment when the cutting tool contacts the anatomical element.

3. The system of claim 2, wherein determining the safety layer comprises determining the distance from the first pose to a second pose based on the dimensional information, the second pose corresponding to when the cutting tool breaches the anatomical element and spacing the safety layer a predetermined distance from the second pose towards the first pose.

4. The system of claim 2, wherein the first pose is received from a robot operating the cutting tool.

5. The system of claim 1, wherein determining the safety layer comprises determining a second surface opposite the first surface based on the dimensional information and spacing the safety layer a predetermined distance from the second surface towards the first surface.

6. The system of claim 5, wherein the predetermined distance is substantially constant throughout the safety layer.

7. The system of claim 5, wherein the predetermined distance is variable through at least a portion of the safety layer.

8. The system of claim 5, wherein spacing the safety layer a predetermined distance from the second surface includes multiplying a distance between the second surface and the first surface by a predetermined safety factor.

9. The system of claim 1, wherein a sensor output is used as part of detecting the cutting tool contacting the anatomical element.

10. The system of claim 9, wherein the sensor output comprises a force measured by a force sensor in response to the cutting tool exerting the force on the force sensor, and wherein the cutting tool contacting the anatomical element is detected when the measured force exceeds a threshold force.

11. The system of claim 1, wherein the dimensional information comprises a three-dimensional model of the anatomical element.

12. The system of claim 1, wherein the memory stores further data for processing by the processor that, when processed, causes the processor to:
generate a notification when the cutting tool contacts the safety layer.

13. A system for determining a safety layer for an anatomical element comprising:
a sensor configured to measure a parameter to yield a sensor output;
a processor; and
a memory storing data for processing by the processor, the data, when processed, causes the processor to:
detect a cutting tool contacting the anatomical element at a first surface using a sensor output received from the sensor;
receive dimensional information of the anatomical element;
determine a safety layer for the anatomical element based on the dimensional information and the detected contact between the cutting tool and the anatomical element at the first surface, wherein the cutting tool is prevented from moving past the safety layer; and
generate a notification when the cutting tool contacts the safety layer.

14. The system of claim 13, wherein the memory stores further data for processing by the processor that, when processed, causes the processor to:
receive information describing a first pose corresponding to a moment when the cutting tool contacts the anatomical element.

15. The system of claim 14, wherein determining the safety layer comprises determining the distance from the first pose to a second pose based on the dimensional data, the second pose corresponding to when the cutting tool breaches the anatomical element and spacing the safety layer a predetermined distance from the second pose towards the first pose.

16. The system of claim 14, further comprising a robotic arm configured to orient the cutting tool, wherein the first pose is received from the robot.

17. The system of claim 13, wherein determining the safety layer comprises determining a second surface opposite the first surface based on the dimensional data and spacing the safety layer a predetermined distance from the second surface towards the first surface.

18. The system of claim 13, wherein the sensor output comprises a force measured by a force sensor in response to the cutting tool exerting the force on the force sensor, and wherein the cutting tool contacting the anatomical element is detected when the measured force exceeds a threshold force.

19. The system of claim 13, wherein the dimensional information comprises a three-dimensional model of the anatomical element.

20. A system for determining a safety layer for an anatomical element comprising:
a robotic arm configured to hold and orient a cutting tool relative to the anatomical element;
a processor; and
a memory storing data for processing by the processor, the data, when processed, causes the processor to:
cause the robotic arm to orient the cutting tool on a trajectory;
detect when the cutting tool contacts the anatomical element at a first surface;
receive dimensional information of the anatomical element;
determine a safety layer for the anatomical element based on the dimensional information and the detected contact, wherein the cutting tool is prevented from moving past the safety layer; and
cause the robotic arm to stop orienting the cutting tool on the trajectory when the cutting tool reaches the safety layer.
